(19) United States
(12) Patent Application Publication
(10) Pub. No.: US 2018/0037806 A1
(43) Pub. Date: Feb. 8, 2018

(54) METHODS AND SYSTEMS EMPLOYING A SULFONATED IMINODIACETIC ACID DURING SUBTERRANEAN TREATMENT OPERATIONS

(71) Applicant: Halliburton Energy Services, Inc., Houston, TX (US)

(72) Inventors: Antonio RECIO, III, Humble, TX (US); Enrique Antonio REYES, Tomball, TX (US); Kevin YORK, Houston, TX (US)

(21) Appl. No.: 15/549,124

(22) PCT Filed: Mar. 4, 2015
(86) PCT No.: PCT/US2015/018627

§ 371 Date: Aug. 4, 2017

(51) Int. Cl. C09K 8/528; E21B 43/26; C07C 229/16; C09K 8/74; C07C 309/14

(52) U.S. Cl. CPC C09K 8/528; C09K 8/74; C07C 309/14; C07C 229/16; E21B 43/26

(57) ABSTRACT

In a subterranean formation, complexation of metal ions can often be desirable in order to mitigate reactivity of the metal ions. However, many chelating agents used for this purpose are commercially manufactured in an alkali metal salt form that may not be entirely suitable for use downhole. The working pH range of some types of chelating agents may also be limiting. Accordingly, methods for treating a subterranean formation can comprise: providing an acidic treatment fluid comprising a sulfonated iminodiacetic acid; introducing the acidic treatment fluid into a subterranean formation; and at least partially dissolving an acid-reactive substance in the subterranean formation while complexing one or more metal ions with the sulfonated iminodiacetic acid.

### BACKGROUND

The present disclosure generally relates to subterranean treatment operations, and, more specifically, to methods and systems for mitigating the presence of metal ions in a subterranean formation during subterranean treatment operations.

Treatment fluids can be used in a variety of subterranean treatment operations. Such treatment operations can include, without limitation, drilling operations, stimulation operations, production operations, remediation operations, sand control treatments, and the like. As used herein, the terms “treat,” “treatment,” “treating,” and grammatical equivalents thereof refer to any subterranean operation that uses a fluid in conjunction with achieving a desired function and/or for a desired purpose. Use of these terms does not imply any particular action by the treatment fluid or a component thereof, unless otherwise specified herein. More specific examples of illustrative treatment operations can include, for example, drilling operations, fracturing operations, gravel packing operations, acidizing operations, scale dissolution and removal operations, sand control operations, consolidation operations, and the like.

Acidic treatment fluids are frequently utilized in the course of conducting various subterranean treatment operations. Illustrative uses of acidic treatment fluids during subterranean treatment operations include, for example, matrix acidizing of siliceous and/or non-siliceous formations, scale dissolution and removal processes, gel breaking, acid fracturing, and the like.

Acidizing operations may be used to stimulate a subterranean formation to increase production of a hydrocarbon resource therefrom. During an acidizing operation, an acid-reactive material in the subterranean formation can be at least partially dissolved by one or more acids to expand existing flow pathways in the subterranean formation, to create new flow pathways in the subterranean formation, and/or to remove acid-soluble scale in the subterranean formation. Acidizing a subterranean formation's matrix can be particularly effective for stimulating production.

The material being reacted with an acidizing fluid can significantly dictate how an acidizing operation is performed. When acidizing a non-siliceous substance, such as a carbonate material, mineral acids, such as hydrochloric acid, may often be sufficient to affect dissolution. Organic acids may be used in a similar manner to hydrochloric acid when dissolving a non-siliceous substance. Siliceous materials, in contrast, are only readily dissolvable using hydrofluoric acid, optionally in combination with other acids to maintain a low pH environment. As used herein, the term “siliceous” refers to a substance having the characteristics of silica, including silicates and/or aluminosilicates. Illustrative siliceous materials can include, for example, silica, silicates, aluminosilicates, and any combination thereof, optionally in further combination with a non-siliceous substance, such as a carbonate material. Most sandstone formations, for example, contain about 40% to about 98% sand quartz particles (i.e., silica), bonded together by various amounts of cementing materials, which may be siliceous in nature (e.g., aluminosilicates or other silicates) or non-siliceous in nature (e.g., carbonates, such as calcite).

Carbonate formations contain minerals that comprise a carbonate anion. Calcite (calcium carbonate) and dolomite (calcium magnesium carbonate) represent illustrative examples. When acidizing a carbonate formation, acidity of the treatment fluid alone can be sufficient to solubilize the carbonate material by decomposing the carbonate anion into carbon dioxide and leaching a metal ion into the treatment fluid. As the concentration of dissolved metal ions rises, particularly at higher pH values upon spending of the acid, the solubility limit may be exceeded and precipitation of scale may occur.

Siliceous formations can include minerals such as, for example, zeolites, clays and feldspars. As indicated above, siliceous formations are usually acidized with hydrofluoric acid, optionally in combination with another acid, in order to react the siliceous minerals and affect their dissolution. Dissolved silicon species can be particularly prone to undergo secondary reactions with alkali metal ions to form highly damaging alkali metal silicate precipitates. Co-present non-siliceous minerals, such as carbonate minerals, may be concurrently dissolved while acidizing a siliceous material.

Calcium ions and other alkaline earth metal ions can be particularly problematic when acidizing either siliceous or non-siliceous formations. As indicated above, the solubility limit of the metal ions may be quickly exceeded and deposition of scale may occur. In the case of siliceous formations acidized with hydrofluoric acid, calcium ions can react readily with free fluoride ions to form highly insoluble calcium fluoride. Other metal ions can prove similarly problematic in this regard. Calcium fluoride and other types of scale formed from metal ions can be highly damaging to subterranean formations, possibly even more so than if the initial acidizing operation had not been performed in the first place.

One approach that has been used to address the issues associated with dissolved metal ions is to employ chelating agents, which can sequester the liberated metal ions in a more soluble and less reactive form of a metal-ligand complex. As used herein, the terms “complex,” “complexing,” “complexation” and other variants thereof refer to the formation of a metal-ligand bond without reference to the mode of bonding. Although complexation of a metal ion may involve a chelation process, complexation is not deemed to be limited in this manner. Once bound in a metal-ligand complex, the metal ions may have a significantly decreased propensity to undergo a further reaction to form scale.

There are difficulties associated with chelation strategies, however. At low pH values, the carboxylic acid groups of many chelating agents may be substantially protonated, a form that can be ineffective for promoting metal ion complexation. This issue can significantly limit the working pH range over which an acidizing operation may take place, potentially limiting the acidizing operation's speed and effectiveness. In addition, many chelating agents are supplied in their alkali metal salt forms, which can be especially problematic for siliceous formations for the reasons noted above. Although a number of chelating agents are known, there may be a very limited number available in suitable form and in sufficient quantities to support various types of subterranean treatment operations.

### BRIEF DESCRIPTION OF THE DRAWINGS

The following figures are included to illustrate certain aspects of the present disclosure and should not be viewed as exclusive embodiments. The subject matter disclosed is capable of considerable modifications, alterations, combinations, and equivalents in form and function, as will occur to one having ordinary skill in the art and the benefit of this disclosure.

FIG. 1 shows an illustrative schematic of a system that can deliver treatment fluids of the present disclosure to a downhole location, according to one or more embodiments.

FIG. 2 shows a bar graph summarizing the amounts of CaCl₂ and NaOH added to the chelant solution of Example 1 and the resulting pH values.

FIG. 3 shows a plot of differential pressure and permeability for a calcite core contacted with the chelant solution of Example 1 as a function of time.

### DETAILED DESCRIPTION

The present disclosure generally relates to subterranean treatment operations, and, more specifically, to methods and systems for mitigating the presence of metal ions in a subterranean formation during subterranean treatment operations.

One or more illustrative embodiments incorporating the features of the present disclosure are presented herein. Not all features of a physical implementation are necessarily described or shown in this application for the sake of clarity. It is to be understood that in the development of a physical implementation incorporating the embodiments of the present disclosure, numerous implementation-specific decisions may be made to achieve the developer's goals, such as compliance with system-related, business-related, government-related and other constraints, which may vary by implementation and from time to time. While a developer's efforts might be time-consuming, such efforts would be, nevertheless, a routine undertaking for one having ordinary skill in the art and the benefit of this disclosure.

As discussed above, there are various issues associated with the use of chelating agents in subterranean acidizing operations. In some instances, their working pH range may be limited. Moreover, the as-supplied alkali metal salt form of many chelating agents can be extremely problematic, particularly when used in conjunction with acidizing a siliceous formation. This issue frequently occurs with aminopolycarboxylic acid chelating agents, which can otherwise be desirable for use in subterranean treatment operations due to their ready complexation properties and environmentally friendly character in many cases. Although the free acid form of a salt-containing chelating agent may be liberated through acidification and ion exchange, these processes may be labor intensive and expensive. Moreover, unless liberated alkali metal ions are removed from a solution of the free acid form of the chelating agent, they can still end up in a formulated treatment fluid, thereby leading to the deleterious consequences noted above.

The present inventors discovered a new type of chelating agent that overcomes several of the above issues and provides other advantages. Namely, the inventors discovered that sulfonated aminopolycarboxylic acids, more specifically sulfonated iminodiacetic acids, can be especially advantageous for use in acidizing operations. As used herein, the term “sulfonated” refers to the condition of a compound bearing a sulfonic acid group. Due to the presence of the sulfonic acid group, sulfonated iminodiacetic acids can maintain high solubility levels and low pH values in aqueous fluids, such as in aqueous acidizing fluids.

Sulfonated iminodiacetic acids can be particularly advantageous in acidizing operations, since they can bear sufficient acidity alone to dissolve an acid-reactive substance, such as a metal carbonate material, without another acid being present for pH control. All of their acidic groups have pKa values under 6, which can allow each acidic group to contribute in acid-promoted dissolution processes to at least some degree. Accordingly, sulfonated iminodiacetic acids can serve as a direct replacement for mineral acids and organic acids that are commonly used in acidizing operations. Such replacement can decrease material costs, transportation expenses and sourcing issues for the additional acids. Optionally, a mineral acid or organic acid can be used in combination with the sulfonated iminodiacetic acids to further promote dissolution of an acid-reactive substance. In particular, when used in conjunction with acidizing a siliceous formation, hydrofluoric acid or a hydrofluoric acid-generating compound can be used in combination with the sulfonated iminodiacetic acids.

Due to their ability to serve as a direct acid replacement, sulfonated iminodiacetic acids may also serve as a secondary acid source in combination with another chelating agent. When combined with one or more other chelating agents, the sulfonated iminodiacetic acid and the other chelating agents may promote complexation of different metal ions and/or provide effectiveness over differing pH ranges.

In spite of their high acidity, sulfonated iminodiacetic acids can advantageously remain active for promoting metal ion complexation and lessen the likelihood of precipitate occurrence. In particular, these compounds can maintain a high affinity for promoting complexation of calcium ions, other alkaline earth metal ions, and transition metal ions. Accordingly, sulfonated iminodiacetic acids can promote dissolution of metal-containing substances through both their acidity and their complexation properties.

The inventors also discovered that certain sulfonated iminodiacetic acids may be readily obtained in their neutral forms, while still being substantially free of alkali metal ions. In contrast to typical synthetic approaches, wherein iminodiacetic acid is alkylated with a haloalkylsulfonic acid under basic conditions to produce an alkali metal salt of the sulfonated iminodiacetic acid, the inventors discovered that sulfonated iminodiacetic acids may be synthesized in their free acid form under acidic conditions via a ring-opening process without alkali metal ions being present. Specifically, iminodiacetic acid may be reacted in high yields with 1,2-oxathietane 2,2-dioxide (ethane sultone), 1,2-oxathiolane 2,2-dioxide (1,3-propane sultone), 1,2-oxathiane 2,2-dioxide (1,4-butane sultone), or various derivatives thereof to accomplish the foregoing. The foregoing sultones are commercially available and directly produce the free acid form of the sulfonated iminodiacetic acid in a fluid concentrate that is substantially free of alkali metal ions. The fluid concentrate may then be employed directly as a treatment fluid or diluted further with a carrier fluid and possibly other substances of interest to form a treatment fluid that is substantially free of alkali metal ions. Sulfonated iminodiacetic acids that are substantially free of alkali metal ions may be particularly advantageous for acidizing a siliceous formation for the reasons discussed above.

The sulfonated iminodiacetic acids described herein may be equally applicable to acidizing operations conducted in a carbonate formation or in a siliceous formation without greatly modifying the treatment fluid. In the case of acidizing a siliceous formation, hydrofluoric acid or a hydrofluoric acid-generating compound can be present in a treatment fluid containing the sulfonated iminodiacetic acid or be introduced separately to a subterranean formation to promote dissolution of the siliceous material therein. The ability to use the sulfonated iminodiacetic acids described herein in both types of acidizing operations may significantly simplify these oilfield operations and decrease their cost. As a further advantage, the sulfonated iminodiacetic acids described herein may possess anti-scaling properties.

In various embodiments, methods described herein may comprise: providing an acidic treatment fluid comprising a sulfonated iminodiacetic acid; introducing the acidic treatment fluid into a subterranean formation; and at least partially dissolving an acid-reactive substance in the subterranean formation while complexing one or more metal ions with the sulfonated iminodiacetic acid.

In some embodiments, the sulfonated iminodiacetic acid may have the structure shown in Formula 1

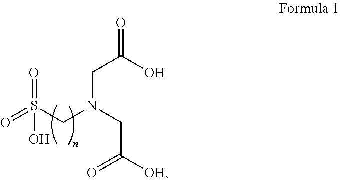

wherein n is an integer ranging between 1 and about 6. Various techniques for synthesizing such sulfonated iminodiacetic acids for use in a treatment fluid are discussed in more detail hereinafter. Additional functionality may also be introduced to the sulfonated iminodiacetic acid of Formula 1 in order to further tailor its solubility, pKa values, and/or biodegradation rate, for example.

In more particular embodiments, n may be 2, 3 or 4 in Formula 1. As referenced above and discussed in more detail below, such sulfonated iminodiacetic acids may be readily synthesized in a form that is substantially free of alkali metal ions.

Any suitable technique can be used for forming the sulfonated iminodiacetic acids of the present disclosure, such as those exemplified by the structure of Formula 1. In some embodiments, iminodiacetic acid or a salt thereof may be alkylated under basic conditions with a haloalkylsulfonic acid or a salt thereof, as shown in Reaction 1, wherein X is a halide or a halide equivalent, such as an alkylsulfonate or an arylsulfonate.

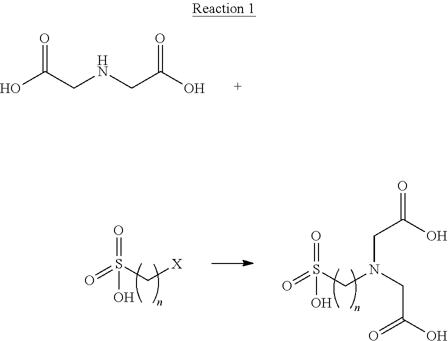

A difficulty with this approach, however, is that metal ions introduced from the base can be difficult to remove. As indicated above, the inventors discovered an alternative approach for synthesizing these compounds by reacting iminodiacetic acid with a sultone under acidic conditions to produce a subset of these compounds in their free acid form and free of alkali metal ions. This approach is exemplified in Reaction 2, wherein A is (CH₂)_{y} and y is 2, 3 or 4.

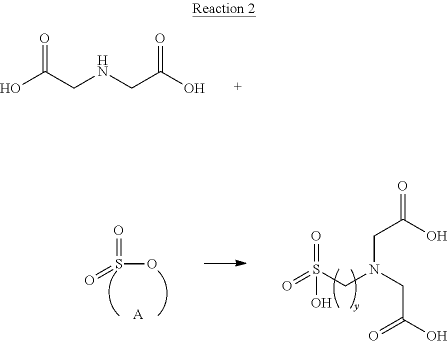

Even to the extent that iminodiacetic acid may be sourced in an alkali metal salt form, the free acid form of the iminodiacetic acid can be produced by acidification, and the alkali metal ions can be removed therefrom via standard techniques before synthesizing the sulfonated iminodiacetic acid by Reaction 2. A further advantage of the synthetic approach depicted in Reaction 2 is that formation of the sulfonated iminodiacetic acid occurs in near-quantitative yield as a concentrate in an aqueous reaction medium. The concentrate may be used directly in an acidizing operation or undergo dilution with a carrier fluid in the course of formulating an acidizing fluid. Accordingly, in some embodiments, the acidic treatment fluids of the present disclosure may be substantially free of alkali metal ions. As used herein, the term “substantially free of alkali metal ions” refers to an alkali metal content of about 5 wt. % or less, particularly an alkali metal content of about 1 wt. % or less.

In further embodiments, providing the acidic treatment fluid may comprise preparing the sulfonated iminodiacetic acid in a form that is substantially free of alkali metal ions and forming the acidic treatment fluid therefrom. The sulfonated iminodiacetic acid may be formed from a sultone in a concentrate, which may be used directly in an acidizing operation or undergo further dilution with a carrier fluid to form an acidizing fluid.

Carrier fluids used for forming the acidic treatment fluids of the present disclosure may be obtained from any suitable source. In some embodiments, suitable aqueous carrier fluids may include, for example, fresh water, acidified water, treated water, salt water, seawater, brackish water, produced water, flowback water, brine (e.g., a saturated salt solution), or an aqueous salt solution (e.g., a non-saturated salt solution). When it is desired for the treatment fluids to be substantially free of alkali metal ions, carrier fluids containing alkali metal ions (e.g., salt water, seawater, brine, aqueous salt water, and the like) may not be suitable, unless they have undergone a treatment to remove a substantial portion of their alkali metal ions.

In further embodiments, an organic co-solvent may be present in the acidic treatment fluids. Suitable organic co-solvents may include, but are not limited to, glycols and alcohol solvents, for example. When present, an amount of the organic co-solvent may range between about 1% to about 50% by volume of the treatment fluid.

In more specific embodiments, preparing the sulfonated iminodiacetic acid in a form that is substantially free of alkali metal ions may comprise reacting iminodiacetic acid with 1,2-oxathiolane 2,2-dioxide (1,3-propane sultone) to form a concentrate comprising the sulfonated iminodiacetic acid, and optionally, diluting the concentrate with a carrier fluid. This reaction forms a —CH₂CH₂— link between the nitrogen atom of the iminodiacetic acid and the sulfonic acid moiety. Related compounds may be formed by reacting ethane sultone or 1,4-butane sultone to form the —CH₂— and —CH₂CH₂CH₂— analogs, respectively. In exemplary embodiments, the reaction to form the sulfonated iminodiacetic acid in this manner may occur at a temperature between about 50° C. and about 100° C., or between about 60° C. and about 80° C.

In some embodiments, the concentrate obtained from the reaction to produce the sulfonated iminodiacetic acid may comprise the acidic treatment fluid directly. In such embodiments, a concentration of the sulfonated iminodiacetic acid in the acidic treatment fluid and in the concentrate may range between about 0.1 M to about 5 M. In more particular embodiments, the concentration may range between about 0.5 M and about 5 M, or between about 1 M and about 5 M, or between about 2 M and about 5 M, or between about 2 M and about 4 M. Dilution of the concentrate with a carrier fluid may be used to lower the concentration of the sulfonated iminodiacetic acid to a suitable working range, such as any concentration in the range of 0.1 M to about 5 M in the treatment fluid.

In some embodiments, the acidic treatment fluid may have a pH of about 4 or below, or a pH of about 3 or below, or a pH of about 2 or below, or a pH of about 1 or below. If the pH of the concentrate or the combination of the concentrate and a carrier fluid is not in a desired pH range, the methods described herein may further comprise raising the pH of the acidic treatment fluid with a base prior to introducing the acidic treatment fluid into the subterranean formation. In some embodiments, ammonium hydroxide may comprise a suitable base, particularly if the treatment fluid is to be maintained in a form that is substantially free of alkali metal ions. Amine bases may also be suitable in this regard. Alkali metal bases may be suitable if there is no need to maintain the acidic treatment fluids free of alkali metal ions. More desirably, however, the acidic treatment fluid may be free of alkali metal ions by employing one or more of the non-metal bases noted above. Similarly, if the pH of the concentrate or the combination of the concentrate and the carrier flow is not low enough, an acid or acid-generating compound may be added thereto, as discussed below.

In more specific embodiments, methods of the present disclosure may comprise: providing an acidic treatment fluid that is substantially free of alkali metal ions and comprises a sulfonated iminodiacetic acid; introducing the acidic treatment fluid into a subterranean formation; at least partially dissolving an acid-reactive substance in the subterranean formation in the presence of the sulfonated iminodiacetic acid; and complexing one or more metal ions in the subterranean formation with the sulfonated iminodiacetic acid.

In the methods of the present disclosure, the sulfonatated iminodiacetic acid may complex one or more metal ions in order to mitigate the metal ions' ability to undergo secondary reactions and form a precipitate within the subterranean formation. The resulting metal complexes may remain within the subterranean formation, or they may be produced therefrom before or concurrent with production of a hydrocarbon resource. In various embodiments, the metal complexes may remain soluble within the acidic treatment fluid. Complexation of metal ions may, at least in part, promote dissolution of an acid-reactive substance.

As indicated above, the sulfonated iminodiacetic acids described herein are highly acidic substances by themselves, and in addition to their metal complexation properties, they may also promote dissolution of the acid-reactive substance containing metal ions through their acidity. Accordingly, in some embodiments, the sulfonated iminodiacetic acid may be present alone in the acidic treatment fluid, such that the acidic treatment fluid is free of mineral acids and other organic acids. In some embodiments, the sulfonated iminodiacetic acid may serve as a replacement for a mineral acid, such as hydrochloric acid, in an acidic treatment fluid. This may particularly be the case when the acidic treatment fluids are used for acidizing a subterranean formation that primarily comprises a carbonate mineral such as calcite, dolomite, or the like.

In some embodiments, however, the acidic treatment fluids containing the sulfonated iminodiacetic acid may further comprise an acid or an acid-generating compound. The acid or acid-generating compound may supplement the innate acidity of the sulfonated iminodiacetic acid so that the treatment fluid spends less quickly. Both organic acids and mineral acids may be used for this purpose. Examples of organic and mineral acids that may be present in the acidic treatment fluids include, for example, hydrochloric acid, hydrobromic acid, hydrofluoric acid formic acid, acetic acid, chloroacetic acid, dichloroacetic acid, trichloroacetic acid, methanesulfonic acid, citric acid, maleic acid, glycolic acid, lactic acid, malic acid, oxalic acid, the like, and any combination thereof. As discussed in more detail herein, it may be particularly desirable to include hydrofluoric acid in combination with the sulfonated iminodiacetic acid when acidizing a subterranean formation comprising a siliceous mineral or a subterranean formation of mixed mineralogy comprising both a siliceous mineral and a carbonate mineral.

Suitable acid-generating compounds can include, for example, esters, aliphatic polyesters, orthoesters, poly(orthoesters), poly(lactides), poly(glycolides), poly(ε-caprolactones), poly(hydroxybutyrates), poly(anhydrides), ethylene glycol monoformate, ethylene glycol diformate, diethylene glycol diformate, glyceryl monoformate, glyceryl diformate, glyceryl triformate, triethylene glycol diformate, formate esters of pentaerythritol, the like, any derivative thereof, and any combination thereof.

In more particular embodiments, the acid-reactive substance may comprise a carbonate mineral. Carbonate minerals that may undergo dissolution in the presence of the acidic treatment fluids of the present disclosure include, for example, calcite (calcium carbonate), dolomite (calcium magnesium carbonate), siderite (iron carbonate), or any combination thereof. Particularly when acidizing a calcite or dolomite formation, the one or more metal ions may comprise at least calcium ions. When acidizing a subterranean formation comprising siderite, the one or more metal ions may comprise iron ions and iron control may be achieved. In either case, by complexing the metal ions with the sulfonated iminodiacetic acid, the solubility limit of the metal ions may be increased relative to the uncomplexed metal ions, thereby lessening the likelihood of damaging precipitates forming within the subterranean formation.

In various embodiments, metal ions that may be complexed by the sulfonated iminodiacetic acid include alkaline earth metal ions, transition metal ions, main group metal ions, lanthanide ions, or any combination thereof. In more particular embodiments, metal ions that may be complexed by the sulfonated iminodiacetic acid may include calcium ions, iron ions, aluminum ions, or any combination thereof. Zinc ions, titanium ions, and/or zirconium ions may also be desirable to complex for similar reasons. By complexing these metal ions and others, it may be possible to preclude their participation in secondary reactions that may form damaging precipitates in a subterranean formation.

In other more particular embodiments, the acid-reactive substance may comprise a siliceous mineral, which optionally may be present in combination with a carbonate mineral. When a siliceous mineral is present, dissolution of the siliceous mineral may be promoted by hydrofluoric acid or a hydrofluoric acid-generating compound. By complexing metal ions that may be present in the subterranean formation, the metal ions may be prevented from undergoing secondary reactions with dissolved silicon compounds or with the hydrofluoric acid itself. For example, by complexing calcium ions, the calcium ions may be prevented from undergoing a secondary reaction to form damaging calcium fluoride precipitates.

Alternately, the one or more metal ions may be co-present with or within the siliceous mineral and be liberated upon dissolution of the siliceous mineral by the hydrofluoric acid. For example, metal ions can be liberated when dissolving a siliceous mineral that is co-present with a carbonate mineral, such as calcite.

When hydrofluoric acid or a hydrofluoric acid-generating compound is used to promote dissolution of a siliceous mineral, the hydrofluoric acid or the hydrofluoric acid-generating compound may be present in the acidic treatment fluid comprising the sulfonated iminodiacetic acid or it may be introduced in a separate treatment fluid. When introduced to the subterranean formation in a separate treatment fluid, the acidic treatment fluid comprising the sulfonated iminodiacetic acid may be introduced to the subterranean formation first in order to promote metal ion complexation before forming dissolved silicon compounds from the siliceous mineral. Alternatively, the acidic treatment fluid comprising the sulfonated iminodiacetic acid may pre-condition the subterranean formation before dissolved silicon is formed therein.

In some embodiments, the hydrofluoric acid may be formed from a hydrofluoric acid-generating compound. Suitable hydrofluoric acid-generating compounds may include substances such as, for example, fluoroboric acid, fluorosulfuric acid, hexafluorophosphoric acid, hexafluoroantimonic acid, difluorophosphoric acid, hexafluorosilicic acid, potassium hydrogen difluoride, sodium hydrogen difluoride, polyvinylammonium fluoride, polyvinylpyridinium fluoride, pyridinium fluoride, imidazolium fluoride, ammonium fluoride, tetrafluoroborate salts, hexafluoroantimonate salts, hexafluorophosphate salts, bifluoride salts (e.g., ammonium bifluoride), perfluorinated organic compounds, boron trifluoride and various boron trifluoride complexes.

When used, a hydrofluoric acid-generating compound can be present in the treatment fluids described herein in an amount ranging between about 0.1% to about 20% by weight of the acidic treatment fluid. In other embodiments, an amount of the hydrofluoric acid-generating compound can range between about 0.5% to about 10% by weight of the treatment fluid or between about 0.5% to about 8% by weight of the treatment fluid. Hydrofluoric acid, when present, may be used in similar concentration ranges.

When a siliceous material is being dissolved by the acidic treatment fluids of the present disclosure, the acidic treatment fluid can be substantially free of alkali metal ions in various embodiments.

In further embodiments, the treatment fluids described herein may further comprise any number of additives that are commonly used in downhole operations including, for example, silica scale control additives, corrosion inhibitors, surfactants, gel stabilizers, anti-oxidants, polymer degradation prevention additives, relative permeability modifiers, scale inhibitors, foaming agents, defoaming agents, antifoaming agents, emulsifying agents, de-emulsifying agents, iron control agents, proppants or other particulates, particulate diverters, acids, fluid loss control additives, gas, catalysts, clay control agents, dispersants, flocculants, scavengers (e.g., H₂S scavengers, CO₂ scavengers or O₂ scavengers), gelling agents, lubricants, friction reducers, bridging agents, viscosifiers, weighting agents, solubilizers, pH control agents (e.g., buffers), hydrate inhibitors, consolidating agents, bactericides, catalysts, clay stabilizers, breakers, delayed release breakers, and the like. Any combination of these additives may be used as well. One of ordinary skill in the art will be able to formulate a treatment fluid having properties suitable for a given application.

In some embodiments, the acidic treatment fluids described herein may be used in treating a particulate pack in a subterranean formation. Particulate packs may include, for example, proppant packs and gravel packs. Treatment of a particulate pack may beneficially allow the permeability of the pack to be increased, such that it presents a lower impediment to fluid flow.

In some embodiments, the acidic treatment fluids described herein may be utilized in matrix acidizing operations. That is, in some embodiments, the treatment fluids described herein can be introduced to a subterranean formation below a fracture gradient pressure of the subterranean formation. In some embodiments, the interaction of the treatment fluid with the formation matrix may result in the desirable formation of wormholes therein. In other embodiments, the treatment fluids described herein can be introduced to a subterranean formation at or above a fracture gradient pressure of the subterranean formation, such that one or more fractures are created or enhanced in the subterranean formation. Given the benefit of the present disclosure and the understanding of one having ordinary skill in the art, one can readily determine whether to introduce the treatment fluids to a subterranean formation at matrix flow rates (i.e., below the fracture gradient pressure) or at fracturing flow rates (i.e., at or above the fracture gradient pressure).

In some or other embodiments, the treatment fluids described herein may be used in remediation operations within a subterranean formation. Specifically, in some embodiments, treatment fluids described herein comprising a sulfonated iminodiacetic acid may be used to remove precipitation or accumulation damage within a subterranean formation. As used herein, the term “precipitation or accumulation damage” refers to a siliceous or non-siliceous scale that has been dissolved in a subterranean formation and deposited elsewhere within the subterranean formation.

In some embodiments, the treatment fluids described herein may be used in conjunction with drilling a wellbore penetrating a subterranean formation. For example, when used during drilling, the treatment fluids may desirably leave the subterranean formation conditioned with the chelating agent so that precipitation can be subsequently mitigated at a later time.

Accordingly, treatment fluids described herein may comprise a sulfonated iminodiacetic acid of Formula 1, in which n is 2, 3, or 4, and the acidic treatment fluid is substantially free of alkali metal ions. In other embodiments, the treatment fluids may further comprise hydrofluoric acid or a hydrofluoric acid-generating compound. In still further embodiments, the treatment fluids may further comprise an acid or an acid-generating compound.

In still other various embodiments, systems configured for delivering a treatment fluid of the present disclosure to a downhole location are described herein. In various embodiments, the systems can comprise a pump fluidly coupled to a tubular, the tubular containing an acidic treatment fluid that is substantially free of alkali metal ions and comprises a sulfonated iminodiacetic acid having the structure exemplified in Formula 1 in which n is 2, 3, or 4.

The pump may be a high pressure pump in some embodiments. As used herein, the term “high pressure pump” will refer to a pump that is capable of delivering a fluid downhole at a pressure of about 1000 psi or greater. A high pressure pump may be used when it is desired to introduce a treatment fluid of the present disclosure to a subterranean formation at or above a fracture gradient of the subterranean formation, but it may also be used in cases where fracturing is not desired. The treatment fluids described herein may be introduced with a high pressure pump, or they may be introduced following a treatment fluid (e.g., a pad fluid) that was introduced with a high pressure pump. In some embodiments, the high pressure pump may be capable of fluidly conveying particulate matter into the subterranean formation. Suitable high pressure pumps will be known to one having ordinary skill in the art and may include, but are not limited to, floating piston pumps and positive displacement pumps.

In other embodiments, the pump may be a low pressure pump. As used herein, the term “low pressure pump” will refer to a pump that operates at a pressure of about 1000 psi or less. In some embodiments, a low pressure pump may be fluidly coupled to a high pressure pump that is fluidly coupled to the tubular. That is, in such embodiments, the low pressure pump may be configured to convey the treatment fluid to the high pressure pump. In such embodiments, the low pressure pump may “step up” the pressure of a treatment fluid before it reaches the high pressure pump.

In some embodiments, the systems described herein can further comprise a mixing tank that is upstream of the pump and in which the sulfonated iminodiacetic acid is provided in a concentrate and/or combined with a carrier fluid. Formation of the sulfonated iminodiacetic acid may also occur in the mixing tank, in some embodiments. In various embodiments, the pump (e.g., a low pressure pump, a high pressure pump, or a combination thereof) may convey the treatment fluid from the mixing tank or other source of the treatment fluid to the tubular. In other embodiments, however, the treatment fluid can be formulated offsite and transported to a worksite, in which case the treatment fluid may be introduced to the tubular via the pump directly from its shipping container (e.g., a truck, a railcar, a barge, or the like) or from a transport pipeline. In either case, the treatment fluid may be drawn into the pump, elevated to an appropriate pressure, and then introduced into the tubular for delivery downhole.

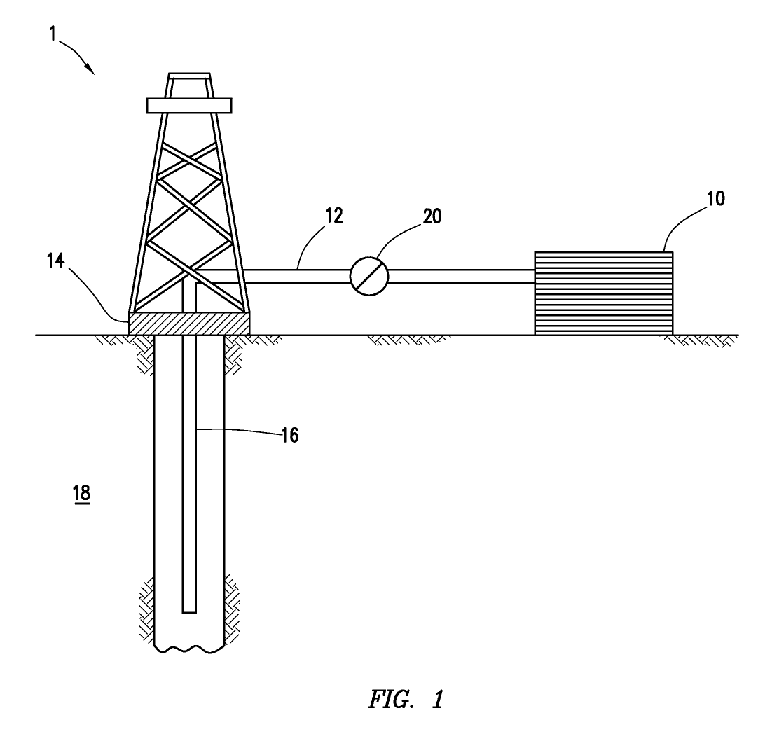

FIG. 1 shows an illustrative schematic of a system that can deliver treatment fluids of the present disclosure to a downhole location, according to one or more embodiments. It should be noted that while FIG. 1 generally depicts a land-based system, it is to be recognized that like systems may be operated in subsea locations as well. As depicted in FIG. 1, system 1 may include mixing tank 10, in which a treatment fluid of the present disclosure may be formulated. The treatment fluid may be conveyed via line 12 to wellhead 14, where the treatment fluid enters tubular 16, tubular 16 extending from wellhead 14 into subterranean formation 18. Tubular 16 may include orifices that allow the treatment fluid to enter into the wellbore. Pump 20 may be configured to raise the pressure of the treatment fluid to a desired degree before its introduction into tubular 16. It is to be recognized that system 1 is merely exemplary in nature and various additional components may be present that have not necessarily been depicted in FIG. 1 in the interest of clarity. Non-limiting additional components that may be present include, but are not limited to, supply hoppers, valves, condensers, adapters, joints, gauges, sensors, compressors, pressure controllers, pressure sensors, flow rate controllers, flow rate sensors, temperature sensors, and the like.

Although not depicted in FIG. 1, the treatment fluid may, in some embodiments, flow back to wellhead 14 and exit subterranean formation 18. Flowback of the treatment fluid may serve to remove complexed metal ions from the subterranean formation, for example. In some embodiments, the treatment fluid that has flowed back to wellhead 14 may subsequently be recovered and recirculated to subterranean formation 18. In other embodiments, the treatment fluid may flow back to wellhead 14 in a produced hydrocarbon fluid from subterranean formation 18.

It is also to be recognized that the disclosed treatment fluids may also directly or indirectly affect the various downhole equipment and tools that may come into contact with the fracturing fluids during operation. Such equipment and tools may include, but are not limited to, wellbore casing, wellbore liner, completion string, insert strings, drill string, coiled tubing, slickline, wireline, drill pipe, drill collars, mud motors, downhole motors and/or pumps, surface-mounted motors and/or pumps, centralizers, turbolizers, scratchers, floats (e.g., shoes, collars, valves, etc.), logging tools and related telemetry equipment, actuators (e.g., electromechanical devices, hydromechanical devices, etc.), sliding sleeves, production sleeves, plugs, screens, filters, flow control devices (e.g., inflow control devices, autonomous inflow control devices, outflow control devices, etc.), couplings (e.g., electro-hydraulic wet connect, dry connect, inductive coupler, etc.), control lines (e.g., electrical, fiber optic, hydraulic, etc.), surveillance lines, drill bits and reamers, sensors or distributed sensors, downhole heat exchangers, valves and corresponding actuation devices, tool seals, packers, cement plugs, bridge plugs, and other wellbore isolation devices, or components, and the like. Any of these components may be included in the systems generally described above and depicted in FIG. 1.

Embodiments disclosed herein include:

A. Methods for acidizing a subterranean formation. The methods comprise: providing an acidic treatment fluid comprising a sulfonated iminodiacetic acid; introducing the acidic treatment fluid into a subterranean formation; and at least partially dissolving an acid-reactive substance in the subterranean formation while complexing one or more metal ions with the sulfonated iminodiacetic acid.

B. Methods for acidizing a subterranean formation. The methods comprise: providing an acidic treatment fluid that is substantially free of alkali metal ions and comprises a sulfonated iminodiacetic acid; introducing the acidic treatment fluid into a subterranean formation; at least partially dissolving an acid-reactive substance in the subterranean formation in the presence of the sulfonated iminodiacetic acid; and complexing one or more metal ions in the subterranean formation with the sulfonated iminodiacetic acid.

C. Acidic treatment fluids comprising a sulfonated iminodiacetic acid. The treatment fluids comprise: a sulfonated iminodiacetic acid having a structure of

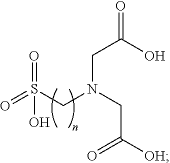

wherein n is 2, 3 or 4; and wherein the acidic treatment fluid is substantially free of alkali metal ions.

D. Systems for introducing a treatment fluid to a subterranean formation. The systems comprise: a pump fluidly coupled to a tubular, the tubular containing an acidic treatment fluid comprising a sulfonated iminodiacetic acid, the sulfonated iminodiacetic acid having a structure of

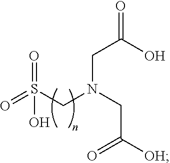

wherein n is 2, 3 or 4; and wherein the acidic treatment fluid is substantially free of alkali metal ions.

Each of embodiments A and B may have one or more of the following additional elements in any combination:

Element 1: wherein the sulfonated iminodiacetic acid has a structure of

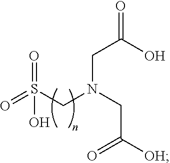

wherein n is an integer ranging between 1 and about 6.

Element 2: wherein n is 2, 3 or 4.

Element 3: wherein the acidic treatment fluid is substantially free of alkali metal ions.

Element 4: wherein the sulfonated iminodiacetic acid also promotes dissolution of the acid-reactive substance.

Element 5: wherein the acid-reactive substance comprises a carbonate mineral.

Element 6: wherein the acidic treatment fluid is free of mineral acids or other organic acids.

Element 7: wherein the acidic treatment fluid further comprises an acid or an acid-generating compound.

Element 8: wherein the acid-reactive substance comprises a siliceous mineral, and hydrofluoric acid or a hydrofluoric acid-generating compound is also present in the subterranean formation; wherein dissolution of the siliceous mineral is promoted by the hydrofluoric acid.

Element 9: wherein the one or more metal ions are co-present with the siliceous mineral and are liberated upon dissolution of the siliceous mineral.

Element 10: wherein the acidic treatment fluid further comprises the hydrofluoric acid or the hydrofluoric acid-generating compound.

Element 11: wherein the one or more metal ions comprise at least calcium ions.

Element 12: wherein the sulfonated iminodiacetic acid has a structure of

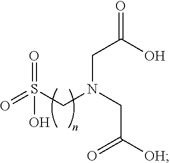

wherein n is 2, 3 or 4.

Element 13: wherein providing the acidic treatment fluid comprises preparing the sulfonated iminodiacetic acid in a form that is substantially free of alkali metal ions, the preparing comprising: reacting iminodiacetic acid with a sultone to form a concentrate comprising the sulfonated iminodiacetic acid; and optionally diluting the concentrate with a carrier fluid.

Element 14: wherein the method further comprises: raising the pH of the acidic treatment fluid with a base prior to introducing the acidic treatment fluid into the subterranean formation.

Each of embodiments C and D may have one or more of the following additional elements in any combination:

Element 15: wherein the treatment fluid further comprises: hydrofluoric acid or a hydrofluoric acid-generating compound.

Element 16: wherein the treatment fluid further comprises: an acid or an acid-generating compound.

Element 17: wherein the treatment fluid is free of mineral acids or other organic acids.

By way of non-limiting example, exemplary combinations applicable to A-D include:

The method of A in combination with elements 1 and 3.

The method of A in combination with elements 1, 2 and 3.

The method of A in combination with elements 4 and 5.

The method of A in combination with elements 1 and 6.

The method of A in combination with elements 8 and 9.

The method of A in combination with elements 3, 8 and 9.

The method of A in combination with elements 8-10.

The method of A in combination with elements 5 and 11.

The method of B in combination with elements 1 and 2.

The method of B in combination with elements 1 and 6.

The method of B in combination with elements 5 and 6.

The method of B in combination with elements 4 and 5.

The method of B in combination with elements 8 and 9.

The method of B in combination with elements 8 and 11.

The method of B in combination with elements 1, 2 and 13.

The treatment fluid of C or the system of D in combination with elements 15 and 16.

To facilitate a better understanding of the embodiments of the present disclosure, the following examples of preferred or representative embodiments are given. In no way should the following examples be read to limit, or to define, the scope of the disclosure.

### EXAMPLES

### Example 1: Synthesis of a Sulfonated Iminodiacetic Acid

15.5 g of iminodiacetic acid (116 mmol) and 14.2 g of 1,2-oxathiolane 2,2-dioxide (116 mmol) were combined in 35 mL water. The reaction mixture was stirred at 85° C. for 4 hours, and reaction progress was monitored by NMR until the resonances of the starting materials disappeared. The resulting aqueous solution was used without purification or further manipulation.

### Example 2: Effects of a Sulfonated Iminodiacetic Acid on Calcium Dissolution

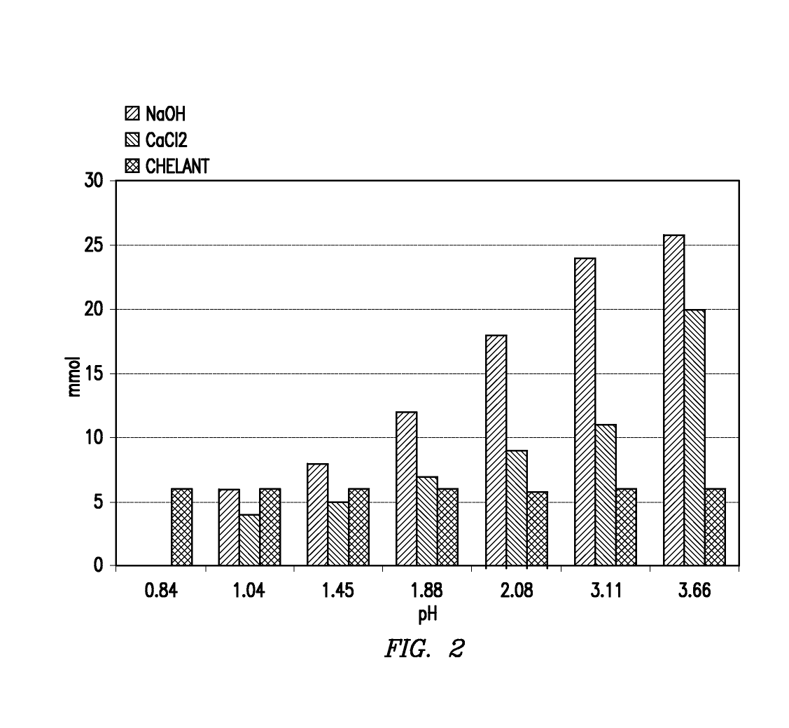

An aliquot of the reaction mixture from Example 1 was diluted to a volume of 10 mL and a concentration of 0.6 M, and the pH was adjusted to approximately 1. Thereafter, varying amounts of CaCl₂ solution and NaOH solution were added to the pH-adjusted aliquot. FIG. 2 shows a bar graph summarizing the amounts of CaCl₂ and NaOH added to the chelant solution of Example 1 and the resulting pH values. No precipitate formation occurred in any of the tested configurations, even at pH values and calcium concentrations where calcium hydroxide precipitation would normally be expected to occur.

### Example 3: Stabilization of Calcium Ions in the Presence of Hydrofluoric Acid

A control solution was prepared by dissolving CaCl₂ and ammonium bifluoride each at a concentration of 0.6 M in 5 mL of water. A test solution was prepared in the same manner as the control solution, except an equivalent molar amount of the chelating agent of Example 1 was added to this solution. The pH value of the control solution was raised to about 7 and the pH value of the test solution was raised to about 4 by adding ammonium hydroxide. Within about 5 minutes, a thick precipitate had formed and settled in the control solution. In contrast, in the test solution, a milky suspension formed, and it did not settle upon standing for 24 hours.

### Example 4: Acidizing of a Calcite Core

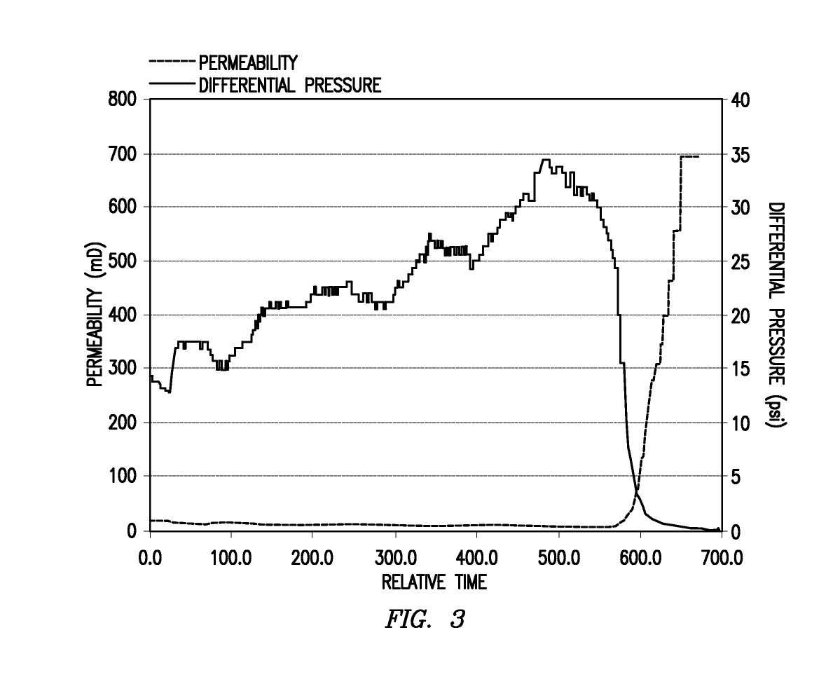

The chelant solution of Example 1 was contacted with a calcite core, and the core's differential pressure and permeability were monitored over time. FIG. 3 shows a plot of differential pressure and permeability for a calcite core contacted with the chelant solution of Example 1 as a function of time. As shown in FIG. 3, a significant drop in differential pressure and an increase in permeability of the core occurred after a contact time period. Thus, it can be concluded that the sulfonated iminodiacetic acid alone was effective to promote dissolution of the calcium carbonate in the core.

Unless otherwise indicated, all numbers expressing quantities of ingredients, properties such as molecular weight, reaction conditions, and so forth used in the present specification and associated claims are to be understood as being modified in all instances by the term “about.” Accordingly, unless indicated to the contrary, the numerical parameters set forth in the specification and attached claims are approximations that may vary depending upon the desired properties sought to be obtained by the embodiments of the present disclosure. At the very least, and not as an attempt to limit the application of the doctrine of equivalents to the scope of the claim, each numerical parameter should at least be construed in light of the number of reported significant digits and by applying ordinary rounding techniques.

Therefore, the present disclosure is well adapted to attain the ends and advantages mentioned as well as those that are inherent therein. The particular embodiments disclosed above are illustrative only, as the present disclosure may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope and spirit of the present disclosure. The disclosure illustratively disclosed herein suitably may be practiced in the absence of any element that is not specifically disclosed herein and/or any optional element disclosed herein. While compositions and methods are described in terms of “comprising,” “containing,” or “including” various components or steps, the compositions and methods can also “consist essentially of” or “consist of” the various components and steps. All numbers and ranges disclosed above may vary by some amount. Whenever a numerical range with a lower limit and an upper limit is disclosed, any number and any included range falling within the range are specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the element that it introduces.

1. A method comprising:
providing an acidic treatment fluid comprising a sulfonated iminodiacetic acid;
introducing the acidic treatment fluid into a subterranean formation; and
at least partially dissolving an acid-reactive substance in the subterranean formation while complexing one or more metal ions with the sulfonated iminodiacetic acid.

2. The method of claim 1, wherein the sulfonated iminodiacetic acid has a structure of 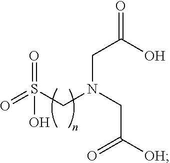
wherein n is an integer ranging between 1 and about 6.

3. The method of claim 2, wherein n is 2, 3 or 4.

4. The method of claim 3, wherein the acidic treatment fluid is substantially free of alkali metal ions.

5. (canceled)

6. The method of claim 5, wherein the acid-reactive substance comprises a carbonate mineral.

7. The method of claim 6, wherein the acidic treatment fluid is free of mineral acids or other organic acids.

8. The method of claim 6, wherein the acidic treatment fluid further comprises an acid or an acid-generating compound.

9. The method of claim 1, wherein the acid-reactive substance comprises a siliceous mineral, and hydrofluoric acid or a hydrofluoric acid-generating compound is also present in the subterranean formation;
wherein dissolution of the siliceous mineral is promoted by the hydrofluoric acid.

10. The method of claim 9, wherein the one or more metal ions are co-present with the siliceous mineral and are liberated upon dissolution of the siliceous mineral.

11. The method of claim 9, wherein the acidic treatment fluid further comprises the hydrofluoric acid or the hydrofluoric acid-generating compound.

12.-13. (canceled)

14. A method comprising:
providing an acidic treatment fluid that is substantially free of alkali metal ions and comprises a sulfonated iminodiacetic acid;
introducing the acidic treatment fluid into a subterranean formation;
at least partially dissolving an acid-reactive substance in the subterranean formation in the presence of the sulfonated iminodiacetic acid; and
complexing one or more metal ions in the subterranean formation with the sulfonated iminodiacetic acid.

15. The method of claim 14, wherein the sulfonated iminodiacetic acid has a structure of 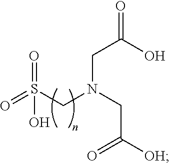
wherein n is 2, 3 or 4.

16. The method of claim 14, wherein providing the acidic treatment fluid comprises preparing the sulfonated iminodiacetic acid in a form that is substantially free of alkali metal ions, the preparing comprising:
reacting iminodiacetic acid with a sultone to form a concentrate comprising the sulfonated iminodiacetic acid; and
optionally diluting the concentrate with a carrier fluid.

17. The method of claim 14, further comprising:
raising the pH of the acidic treatment fluid with a base prior to introducing the acidic treatment fluid into the subterranean formation.

18. (canceled)

19. The method of claim 18, wherein the acid-reactive substance comprises a carbonate mineral.

20. The method of claim 19, wherein the acidic treatment fluid is free of mineral acids or other organic acids.

21. The method of claim 19, wherein the acidic treatment fluid further comprises an acid or an acid-generating compound.

22. The method of claim 14, wherein the acid-reactive substance comprises a siliceous mineral, and hydrofluoric acid or a hydrofluoric acid-generating compound is also present in the subterranean formation;
wherein dissolution of the siliceous mineral is promoted by the hydrofluoric acid.

23. The method of claim 22, wherein the one or more metal ions are co-present with the siliceous mineral and are liberated upon dissolution of the siliceous mineral.

24.-26. (canceled)

27. An acidic treatment fluid comprising:
a sulfonated iminodiacetic acid having a structure of 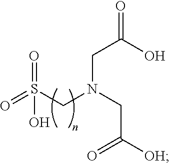
wherein n is 2, 3 or 4; and
wherein the acidic treatment fluid is substantially free of alkali metal ions.

28.-30. (canceled)
